SEQUENCE LISTING (19 sequences)

SEQ ID NO: 1 - DNA, 21 bp, Artificial Sequence
  Chemically Synthesized
caatttgatccaccgactgtt

SEQ ID NO: 2 - DNA, 21 bp, Artificial Sequence
  Chemically Synthesized
ccgcatgaatcttactctgga

SEQ ID NO: 3 - DNA, 21 bp, Artificial Sequence
  Chemically Synthesized
ggatgcttatgaataatccag

SEQ ID NO: 4 - DNA, 20 bp, Artificial Sequence
  Chemically Synthesized
gtataggattgccattttgg

SEQ ID NO: 5 - DNA, 47 bp, Artificial Sequence
  Chemically Synthesized
gaattaatacgactcactatagggagaccaaagttggaagggaatga

SEQ ID NO: 6 - DNA, 48 bp, Artificial Sequence
  Chemically Synthesized
gaattaatacgactcactatagggagaccgcatgaatcttactctgga

SEQ ID NO: 7 - DNA, 48 bp, Artificial Sequence
  Chemically Synthesized
gaattaatacgactcactatagggagaggatgcttatgaataatccag

SEQ ID NO: 8 - DNA, 47 bp, Artificial Sequence
  Chemically Synthesized
gaattaatacgactcactatagggagagtataggattgccattttgg

SEQ ID NO: 9 - DNA, 45 bp, Artificial Sequence
  Chemically Synthesized
gaattaatacgactcactatagggagatgaaagctggctacagga

SEQ ID NO: 10 - DNA, 45 bp, Artificial Sequence
  Chemically Synthesized
gaattaatacgactcactatagggagagcaggcttctgcttcaat

SEQ ID NO: 11 - DNA, 491 bp, Halyomorpha halys
gttaaactaggtggctgacaagaaaaagactcgcgactagttcattcttaagctagacaccgcggagtaagtaaactccaacacctccttctataagaagcgatctcgactaactacttgagagtgatctcctacttctcgaactcctgcttcttcgaggaacagattaacccaatatggacaaaaggagtctgcttgatgatctcgatcaactaagcgaacttgacgacaacccaactccccctccacaacgactagaacaaaaagagaagtatgaaactaaggtaaataaaagaagtgatgacctccttctcgaactcctgcttctacttcttctgctacttcaacttagtcttggaagcgatgatagtggtaaaacacctataggtataagtataagcatcgggtttaataatttgcggaatcctgataaatacgagtccagacttggaaacaccccgtgatggtataggtctcattctaagtacgcc

SEQ ID NO: 12 - DNA, 499 bp, Halyomorpha halys
ggatgcttatgaataatccagagctgtatacaaatgtaaatgcattgcaaaaacgcgatgcacaagaggtcttggaagaagttaaagatctattaccatggtctataggagaaaacgtgctagatgttggctgtggacctggtgatctcacatcctcccttctcacttcatatctggccaatgactatcgagtggtcggttgcgatatttctgaagctatggtgaaatatgctcaagcaaaatatggaaacgatcaattttgtttcaaacagcttgatatcagcaatggaaatatatggatgaactgggaagaggagatttttgataaagtattttcattttactgccttcactgggttaaagatcagatacaagcagcagaaaacatttatagtttgctgaaagatggtggttattttgtcacaatgttcactatatctcatccgtttcttattctatttagcagacttaaggaaaacccaaaatggcaatcctatac

SEQ ID NO: 13 - DNA, 595 bp, Escherichia coli
tgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgc

SEQ ID NO: 14 - DNA, 26 bp, Artificial Sequence
  Chemically Synthesized
ttgatagttgtttggattttgaaggt

SEQ ID NO: 15 - DNA, 23 bp, Artificial Sequence
  Chemically Synthesized
tcttacttgatcagcgctcagaa

SEQ ID NO: 16 - DNA, 21 bp, Artificial Sequence
  Chemically Synthesized
aggaaaacccaaaatggcaat

SEQ ID NO: 17 - DNA, 30 bp, Artificial Sequence
  Chemically Synthesized
atgtattcttcttttggatcttttcttgag

SEQ ID NO: 18 - DNA, 21 bp, Artificial Sequence
  Chemically Synthesized
atgcccccgcctgtccttatt

SEQ ID NO: 19 - DNA, 21 bp, Artificial Sequence
  Chemically Synthesized
tgaaagcagcctgaatagtgg

(19) United States
(12) Patent Application Publication
(10) Pub. No.: US 2018/0023076 A1
(43) Pub. Date: Jan. 25, 2018

(54) DOUBLE STRAND RNA DELIVERY SYSTEM FOR PLANT-SAP-FEEDING INSECTS

(71) Applicant: The United States of America, as Represented By the Secretary of Agriculture, Washington, DC (US)

(72) Inventors: SAIKAT KUMAR B. GHOSH, Ellicott City, MD (US); DAWN E. GUNDERSEN-RINDAL, SILVER SPRING, MD (US); WAYNE B. HUNTER, PORT ST. LUCIE, FL (US)

(21) Appl. No.: 15/218,366

(22) Filed: Jul. 25, 2016

(51) Int. Cl. C12N 15/113; A01N 43/16

(52) U.S. Cl. CPC C12N 15/113; A01N 43/16; C12N 2310/14; C12N 2320/50

(57) ABSTRACT

The present disclosure provides compositions and methods of delivering double strand ribonucleic acid (dsRNA) to insects that penetrate plant tissues to feed on sap and other liquid components of plants. Taking advantage of the liquid transport capabilities of plant vascular structures, dsRNA is provided to plant tissues in an aqueous solution that is then transported throughout the tissues. The dsRNA-laden plant material is then presented to insects that can ingest the dsRNA by feeding on the plant tissue.

### BACKGROUND OF THE INVENTION

### Field of Invention

The present disclosure provides compositions and methods of delivering double strand ribonucleic acid (dsRNA) to insects that penetrate plant tissues to feed on plant sap (in either xylem or phloem) and other liquid components of plants. Taking advantage of the liquid transport capabilities of plant vascular structures, dsRNA is provided to plant tissues in an aqueous solution that is then transported throughout the tissues. The dsRNA-laden tissue is then presented to insects that can ingest the dsRNA by feeding on the plant tissue. Thus, provided herein is a mechanism that allows for dispersal of insect-controlling dsRNA species to insect pests without the time and expense of creating transgenic plants.

### Background

Insect pests around the world are the most extensive group of animals adversely affecting urban and rural plants and other animals. Halyomorpha halys (Stål) (Heteroptera: Pentatomidae), the brown marmorated stink bug (BMSB), is an example of such an invasive insect pest, and it poses a significant ecological and economic impact of billions of dollars collectively. In, or around, 1989, this new invasive insect pest from Asia (China, Taiwan, Korea, and Japan) was accidentally introduced into Allentown, Pa. (Xu et al., Biol. Invasions, (2014)16:153-66). BMSB is a polyphagous piercing/sucking insect that feeds on hundreds of known plant hosts including specialty crops such as apples, stone and pome fruits, grapes, ornamental plants, vegetables, seed crops, as well as such staple crops as soybean and corn. BMSB has been detected in 42 states in the United States, Canada and Europe, and its damage has been predominantly in the Mid-Atlantic Region (DE, MD, PA, NJ, VA, and WV) (Leskey et al., Outlooks Pest Mgmt., (2012) 23:218-26). Along with crop damage BMSB can elicit allergic reactions leading to conjunctivitis and rhinitis in individuals sensitive to aeroallergens or contact dermatitis upon exposure to the crushed animal (Anderson et al., Dermatitis (2012) 23:170-72; Mertz et al., J. Allergy Clin. Immunol. (2012) 130:999-1001). This invasive insect pest is also a nuisance and can be attracted in large numbers in structures such as houses, schools and other indoor spaces that provide a safe hiding area in the fall to overwinter until spring for mating and egg laying (Lesky et al., supra). Few non-chemical control methods have been discovered, leading us to investigate the possibility of using RNA-mediated interference (RNAi) as an approach to control this, and other, sap-feeding insects.

The discovery of RNAi has facilitated research to understand gene function and regulation. RNAi is a well described gene regulatory mechanism wherein exogenous dsRNA is introduced into the cells of eukaryotic organisms and targets degradation of host cell mRNAs containing sequences complementary to the dsRNA (Mello and Conte, Nature (2004) 431:338-42). RNAi depletes host mRNA either by transcriptional gene silencing, or at a posttranscriptional level thereby affecting translation of the protein (Ambros, Nature (2004) 431:350-55). RNAi takes advantage of internal cellular defenses against the presence of dsRNA, which typically indicates an on-going viral infection. Double stranded RNAs (dsRNA) are cleaved by Dicer, a member of the RNase III superfamily of bidentate nucleases that are evolutionarily conserved in worms, flies, plants, fungi and mammals (Bernstein et al., Nature (2001) 409:6818, 363-66; Ketting et al., Genes Develop, (2001) 15:20, 2654-59; Macrae et al., Science (2006) 311:195-98). These 19-21 base pair short RNAs or siRNAs, unwind and together with RNA-induced silencing complex (RISC) associate with the complementary RNA. This RISC-RNA complex in conjunction with argonaute multi-domain protein containing an RNAse H like domain is responsible for target degradation and silencing the gene (Martinez et al., Cell, (2002) 110:5, 563-74; Bartel, Cell, (2004) 116:2, 281-97).

Double strand RNA was first introduced in to C. elegans by way of microinjection (Fire et al., Nature (1998) 391:806-11) to knockdown the unc-22 gene. This was followed by another report of RNAi using microinjection in D. melanogaster to ablate the frizzled genes (Kennerdell and Carthew, Cell (1998) 95:1017-26). Subsequently, many RNAi effectors have been reported where the dsRNA was delivered by microinjection. dsRNA was dorsally injected in the middle of L3 abdomen of immobilized pea aphid (Acyrthosiphon pisum) (Jaubert-Possamai et al., BMC Biotech. (1998) 7:63), while in honeybee (Apis mellifera) the site of injection was made dorsally between the 5th and 6th abdominal segment and to the eggs (Amadam et al., BMC Biotech. (2003) 3:1). Araujo and colleagues (Araujo et al., Insect Biochem. Mol. Biol. (2006) 36:683-93) noted that dsRNA delivery by microinjection or ingestion to the nymphs of traitomine bug (Rhodnius prolixus) showed depletion of the nitrophorin 2 gene. Albeit dsRNA delivered by ingestion was less traumatic to the insects and the insects remained healthier than the counterparts (Araujo et al., supra; Wuriyanghan et al., PLoS ONE (2011) 6:e27736). Such non-sterile septic punctures have shown to elicit increased expression of immune-related genes in BMSB (Sparks et al., PLoS ONE, (2014) 9:e111646). Delivery of dsRNAs by injection is not only tedious and impracticable for a successful bio-pesticide but may also represent mortality due to trauma at the site of injection than RNAi.

Mello and colleagues reported pos-1 embryonic lethal phenotype in the F1 progeny of C. elegans by simply soaking the worms in dsRNA to induce specific interference (Fire et al., supra). While another study demonstrated RNAi by delivering gus-dsRNA to D. melanogaster neonates by simply soaking them in a solution containing species-specific dsRNA. Additionally, mortality in 4 different species of Drosophila was also reported when dsRNA was delivered by feeding tubulin-dsRNA to these animals while species-specific insecticidal effects were reported when vATPase-dsRNA was orally-delivered to different insect species that included flour beetle (Tribolium castaneum), pea aphid (A. pisum) and tobacco hornworm (M. sexta) (Whyard et al., Insect Biochem. Mol. Biol. (2009) 39:824-32). Another recent study successfully demonstrated depletion of multiple genes in potato/tomato psyllid (Bactericerca cockerelli) using an artificial diet facilitated delivery protocol (Wuriyanghan et al., supra).

One of the first bioassays demonstrating RNAi through oral ingestion of dsRNA was demonstrated in the Western corn rootworm (WCR) (Diabrotica virgifera virgifera). WCR specific dsRNA was applied to an artificial diet of WCR agar for feeding the animals. Numerous dsRNAs were identified that depleted the specific genes resulting in larval stunting as well as mortality (Baum et al., Nat. Biotech. (2007) 25:1322-26). Another novel method of delivering dsRNA was demonstrated through a nanoparticle-mediated depletion of target RNA in mosquitoes. Chitosan was used to produce stable chitosan-dsRNA nanoparticles through electrostatic interaction and delivered to mosquitoes through artificial diet for successful RNAi (Zhang et al., Insect Mol. Biol. (2010) 19:683-93).

Despite successful experiments reported using dsRNA mediated RNAi, to utilize RNAi in agriculture pest management, the most practical route of delivery of dsRNA must be through oral ingestion into the insect. The foremost challenge in sap-feeding insects has been the delivery of dsRNA to these animals. Transgenic plants expressing species-specific dsRNA were used to silence genes in the cotton bollworm and WCR indicating a steady progress towards RNAi technology (Baum et al., supra; Mao et al., Nat. Biotech. (2007) 25:1307-13).

Effecting agriculturally relevant insect-controlling RNAi in plant-sap-feeding insects (e.g., BMSB), requires dsRNA uptake by the targeted insects via presentation in vivo through the vascular tissue of the plant. As discussed above, currently available methodologies to introduce dsRNA to such insects is economically infeasible, technically difficult, or both. As such, we disclose herein inexpensive compositions and methodologies to target sap-feeding insects in agricultural settings, as well as direct and inexpensive methodologies and compositions to test dsRNA molecules that can effectively target such insects without the time, expense and difficulty of creating transgenic plants expressing sufficient levels of dsRNA. This delivery protocol is certainly of importance for the continued development of biomolecular pest management.

### SUMMARY OF THE INVENTION

In one embodiment of the invention disclosed herein, this application provides a composition comprising a living plant material and at least one double-strand RNA (dsRNA) not produced by the living plant material, where the at least one dsRNA is distributed throughout at least part of the living plant material's vascular tissues and where the living plant material does not contain genetic information allowing for the production of the at least one double strand dsRNA. In some instances, the living plant material is a fruit, vegetable, stem or leaf such as a green bean or collard green leaf. In some embodiments, the dsRNA is capable of interfering with polypeptide production in at least one insect. In preferred embodiments, the insect is a sap-feeding insect, including xylem sap and phloem sap. In particular embodiments, the insect is a brown marmorated stink bug, a harlequin bug or a pea aphid. In some instances, there are two or more distinct dsRNA species. The dsRNA present in the plant materials of the present invention can be at a concentration of 1-2 μg per inch of the living plant material. The dsRNA can be introduced to the living plant material by soaking a portion of the living plant material in an aqueous solution comprising the dsRNA. This aqueous solution can contain one or more dsRNA species at a concentration of 2-10 μg/ml.

Further provided herein is a method of inducing RNA interference (RNAi) in an insect, comprising the steps of: a) providing a living plant material containing a dsRNA not produced by the living plant material, where the dsRNA is distributed throughout at least part of the living plant material's vascular tissues and where the living plant material does not contain genetic information allowing for the production of the dsRNA; and b) allowing the insect to ingest a sufficient amount of the dsRNA by feeding on the plant material to interfere with the production of at least one protein targeted by the at least one dsRNA, thereby inducing RNAi in the insect. In some instances the dsRNA is present at a concentration of 1-2 μg per inch of the living plant material. The living plant material can be a fruit, vegetable, stem or leaf, such as a green bean or collard green leaf. In some embodiments, the induced RNAi is at a level to control the insect. In preferred embodiments, the insect is a plant sap-feeding insect, including xylem sap and phloem sap. In specific embodiments the insect is a brown marmorated stink bug, a harlequin bug or a pea aphid. The dsRNA utilized can comprise two or more distinct dsRNA species, for example, two dsRNA species that target protein production in two or more insects. The dsRNA can be introduced to the living plant material by soaking a portion of the living plant material in an aqueous solution comprising the dsRNA. This aqueous solution can contain one or more dsRNA species at a concentration of 2-10 μg/ml. In some embodiments of this methodology, the living plant material is provided to the insect within six days of introducing the at least one dsRNA into the living plant material.

In yet another embodiment, herein provided is a method of controlling an insect comprising the steps of: a) providing a living plant material containing a dsRNA not produced by the living plant material, where the dsRNA is distributed throughout at least part of the living plant material's vascular tissues and where the living plant material does not contain genetic information allowing for the production of the dsRNA; b) allowing the insect to ingest a sufficient amount of the dsRNA by feeding on the plant material to interfere with the production a protein targeted by the dsRNA, thereby inducing RNAi in the insect, and; c) controlling the insect via RNAi. In some instances the dsRNA is present at a concentration of 1-2 μg per inch of the living plant material. The living plant material can be a fruit, vegetable, stem or leaf, such as a green bean or collard green leaf. In some embodiments, the induced RNAi is at a level to control the insect. In preferred embodiments, the insect is a plant sap-feeding insect, including xylem sap and phloem sap. In specific embodiments the insect is a brown marmorated stink bug, a harlequin bug or a pea aphid. The dsRNA utilized can comprise two or more distinct dsRNA species, for example, two dsRNA species that target protein production in two or more insects. The dsRNA can be introduced to the living plant material by soaking a portion of the living plant material in an aqueous solution comprising the dsRNA. This aqueous solution can contain one or more dsRNA species at a concentration of 2-10 μg/ml. In some embodiments of this methodology, the living plant material is provided to the insect within six days of introducing the at least one dsRNA into the living plant material.

### INCORPORATION BY REFERENCE

All publications, patents and patent applications mentioned in this specification are herein incorporated by reference to the same extent as if each individual publication, patent or patent application was specifically and individually indicated to be incorporated by reference.

### BRIEF DESCRIPTION OF THE DRAWINGS

The novel features of the invention are set forth with particularity in the claims. Features and advantages of the present invention are referred to in the following detailed description, and the accompanying drawings of which:

FIGS. 1A-1D provide pictorial depictions of delivery of nutrients through green beans. FIG. 1A: Organic green beans were washed with sodium hypochlorite and trimmed from the calyx end to a total length of 3 inches. These beans were then immersed in ddH₂O or ddH₂O solution with green food color for a period of 3 hrs. Transport of green food color was observed in the green bean encircled by the oval area at the exposed calyx. FIG. 1B: BMSB feeding bioassay. Three animals were placed in a magenta jars with 3 greens beans immersed in either 2 ml microcentrifuge containing ddH₂O or a solution of ddH₂O and green food color. FIG. 1C: BMSB placed in the magenta jars are able to pierce through the green beans and reach the diet with their stylets. FIG. 1D: BMSB frass observed on day 2 and 3 of ingesting a solution of ddH₂O and green food color through green beans.

FIGS. 2A-2F provide a comparison of natural and artificial diets. Six diets were compared to assess optimal rearing of BMSB. Each magenta jar contained one BMSB and the test diet that are: (FIG. 2A) BMSB reared on green beans; (FIG. 2B) BMSB reared on green beans immersed in 300 μl of ddH₂O; (FIG. 2C) BMSB reared on artificial gypsy moth diet; (FIG. 2D) BMSB reared on artificial diet formulated for BMSB consisting of 2% agar, (FIG. 2E) BMSB reared on artificial diet formulated for BMSB consisting of 8% agar, and; (FIG. 2F) BMSB reared on artificial diet formulated for BMSB consisting of green bean puree.

FIGS. 3A-3F provide a comparison of the effects of various diets on BMSB nymph growth. BMSB nymphs 5 each were allowed to feed for a period of 4 weeks during which their body masses were recorded. The data was plotted for individual animals using KALEIDAGRAPH (Synergy software) for their respective diets, as indicated: (FIG. 3A) BMSB reared on green beans; (FIG. 3B) BMSB reared on green beans immersed in 300 μl of ddH₂O; (FIG. 3C) BMSB reared on artificial gypsy moth diet; (FIG. 3D) BMSB reared on artificial diet formulated for BMSB consisting of 2% agar; (FIG. 3E) BMSB reared on artificial diet formulated for BMSB consisting of 8% agar and; (FIG. 3F) BMSB reared on artificial diet formulated for BMSB consisting of green bean puree.

FIG. 4 depicts analysis of dsRNA delivered through green beans. dsRNA of the E. coli LacZ gene (lane 2), the BMSB Juvenile Hormone gene (JH) (lane 3), and the BMSB Vitellogenin (Vg) (lane 4), were obtained after PCR products from genomic DNA were amplified with primers containing T7 promoter sequence. These fragments were further transcribed using T7 polymerase; the obtained in vitro transcribed dsRNA was confirmed by electrophoresis on 1% agarose and visualized by staining with SYBR GOLD (Life technologies) alongside a DNA ladder (Lane 1). Green beans were immersed in 5 μg of dsRNA for 1 day and fragments of LacZ gene (lane 6), JH (lane 7), and Vg (lane 8), were obtained after PCR products of total RNA isolated from the green bean used for delivering dsRNA was amplified and confirmed by electrophoresis on 1% agarose and visualized by staining with Sybr Gold (Life technologies) alongside a DNA ladder (Lane 5). Green beans were immersed in 5 gig of dsRNA for 6 days fragments of LacZ gene (lane 10), JH (lane 11), and Vg (lane 12), were obtained after PCR products of total RNA isolated from the green bean used for delivering dsRNA was amplified and confirmed by electrophoresis on 1% agarose and visualized by staining with SYBR GOLD (Life Technologies) alongside a DNA ladder (Lane 9).

FIGS. 5A-5C provides graphs depicting relative transcript levels of several genes targeted by dsRNA delivered to BMSB nymphs via green beans as measured by quantitative RT-PCR. Total RNA from BMSB 4^{th} instar nymphs fed on JH (FIG. 5A) 5 μg, (FIG. 5B) 20 μg and Vg (FIG. 5C) 5 μg dsRNAs delivered through green beans was isolated and the levels of transcripts were measured by qPCR. LacZ RNAi (mock) served as a negative control. 18s RNA was used as an internal standard to correct for differences in RNA recovery from tissues. Results are from three biological replicates, and error bars indicate SEM.

FIGS. 6A-6H provide depictions of harlequin bug (Murgantia histrionica) feeding on green beans. FIG. 6A: Organic green beans were washed with sodium hypochlorite and trimmed from the calyx end to a total length of 3 inches. These beans were then immersed in ddH₂O or ddH₂O solution with green food color for a period of 3 hrs. Three each of 4^{th} instar HB nymph were allowed to resume feeding on these beans after 24 hr starvation in each magenta vessel. FIG. 6B: HB feeding bioassay day 2. FIG. 6C & FIG. 6D: Some animals were observed to molt on day 3 of feeding. FIG. 6E & FIG. 6F: Animals were observed to be feeding on green beans on days 3 and 4 respectively. FIG. 6G & FIG. 6H: Green colored frass was observed on day 5 and 6 of feeding HB with green beans immersed in water and green food color.

FIGS. 7A-E provide depictions of pea aphid (A. pisum) feeding on green beans. FIG. 7A: Organic green beans were washed with sodium hypochlorite and trimmed from the calyx end to a total length of 3 inches. These beans were then immersed in ddH₂O or ddH₂O solution with green food color for a period of 3 hrs. Fifteen animals each of pea aphids were allowed to resume feeding on these beans after a 24 hr starvation. FIG. 7B & FIG. 7C: Pea aphid feeding bioassay. Animals feeding on beans immersed in either ddH₂O or a solution of ddH₂O and green food color respectively. FIG. 7D: Frass was barely observed on day 3 of feeding pea aphids with green beans immersed in water. FIG. 7E: Green colored frass observed on day 3 of feeding pea aphids with green beans immersed in water.

FIGS. 8A-F provide depictions of harlequin bug (M. histrionica) feeding on baby collard greens. FIG. 8A: Organic grown baby collard greens of approximately 3-4 inch length were washed with sodium hypochlorite. The petioles of these leaves were then immersed in ddH₂O or ddH₂O solution with green food color for a period of 3 hrs. Three each of 4^{th} instar HB nymph were allowed to resume feeding on these beans after 24 hr starvation in each magenta vessel. FIG. 8B: HB feeding bioassay day 1 containing ddH₂O. FIG. 8C: HB feeding bioassay day 1 containing ddH₂O solution with green food color. FIG. 8D: Day 3 of feeding. FIG. 8E: Frass was observed on day 3 of feeding HB with green beans immersed in water. FIG. 8F: Green frass was observed on day 3 of feeding HB with green beans immersed in water and green food color.

### DETAILED DESCRIPTION OF THE INVENTION

Preferred embodiments of the present invention are shown and described herein. It will be obvious to those skilled in the art that such embodiments are provided by way of example only. Numerous variations, changes, and substitutions will occur to those skilled in the art without departing from the invention. Various alternatives to the embodiments of the invention described herein may be employed in practicing the invention. It is intended that the included claims define the scope of the invention and that methods and structures within the scope of these claims and their equivalents are covered thereby.

Technical and scientific terms used herein have the meanings commonly understood by one of ordinary skill in the art to which the instant invention pertains, unless otherwise defined. Reference is made herein to various materials and methodologies known to those of skill in the art. Standard reference works setting forth the general principles of recombinant DNA technology include Sambrook et al., “Molecular Cloning: A Laboratory Manual”, 2d ed., Cold Spring Harbor Laboratory Press, Plainview, N.Y., 1989; Kaufman et al., eds., “Handbook of Molecular and Cellular Methods in Biology and Medicine”, CRC Press, Boca Raton, 1995; and McPherson, ed., “Directed Mutagenesis: A Practical Approach”, IRL Press, Oxford, 1991.

Any suitable materials and/or methods known to those of skill can be utilized in carrying out the instant invention. Materials and/or methods for practicing the instant invention are described. Materials, reagents and the like to which reference is made in the following description and examples are obtainable from commercial sources, unless otherwise noted.

Disclosed here are specific insect pest dsRNA constructs that target several H. halys and other sap-feeding insect gene products. Using dsRNA inhibiting expression of the the disclosed genes as a means of interfering with critical functions of the gene products, a novel method for pest management is disclosed, as well as new products to control certain insect pests.

### Definitions

As used in the specification and claims, use of the singular “a”, “an”, and “the” include plural references unless the context clearly dictates otherwise.

The terms isolated, purified, or biologically pure as used herein, refer to material that is substantially or essentially free from components that normally accompany the referenced material in its native state.

The term “about” is defined as plus or minus ten percent of a recited value. For example, about 1.0 g means from a range of 0.9 g to 1.1 g and all values within that range, whether specifically stated or not.

The term “gene” refers to a DNA sequence involved in producing a RNA or polypeptide or precursor thereof. The polypeptide or RNA can be encoded by a full-length coding sequence or by intron-interrupted portions of the coding sequence, such as exon sequences.

The term “oligonucleotide” refers to a molecule comprising a plurality of deoxyribonucleotides or ribonucleotides. Oligonucleotides may be generated in any manner known in the art, including chemical synthesis, DNA replication, reverse transcription, polymerase chain reaction, or a combination thereof. In one embodiment, the present invention embodies utilizing the oligonucleotide in the form of dsRNA as means of interfering with a critical developmental or reproductive process that leads to control. Inasmuch as mononucleotides are synthesized to construct oligonucleotides in a manner such that the 5′ phosphate of one mononucleotide pentose ring is attached to the 3′ oxygen of its neighbor in one direction via a phosphodiester linkage, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends.

When two different, non-overlapping oligonucleotides anneal to different regions of the same linear complementary nucleic acid sequence, and the 3′ end of one oligonucleotide points towards the 5′ end of the other, the former may be called the “upstream” oligonucleotide and the latter the “downstream” oligonucleotide.

The term “primer” refers to an oligonucleotide, which is capable of acting as a point of initiation of synthesis when placed under conditions in which primer extension is initiated. An oligonucleotide “primer” may occur naturally, as in a purified restriction digest or may be produced synthetically.

A primer is selected to be “substantially complementary” to a strand of specific sequence of the template. A primer must be sufficiently complementary to hybridize with a template strand for primer elongation to occur. A primer sequence need not reflect the exact sequence of the template. For example, a non-complementary nucleotide fragment may be attached to the 5′ end of the primer, with the remainder of the primer sequence being substantially complementary to the strand. Non-complementary bases or longer sequences can be interspersed into the primer, provided that the primer sequence is sufficiently complementary with the sequence of the template to hybridize and thereby form a template primer complex for synthesis of the extension product of the primer.

As used herein, “dsRNA” refers to double-stranded RNA that comprises a sense and an antisense portion of a selected target gene (or sequences with high sequence identity thereto so that gene silencing can occur), as well as any smaller double-stranded RNAs formed therefrom by RNAse or dicer activity. Such dsRNA can include portions of single-stranded RNA, but contains at least 19 nucleotides double-stranded RNA. In one embodiment of the invention, a dsRNA comprises a hairpin RNA which contains a loop or spacer sequence between the sense and antisense sequences of the gene targeted, preferably such hairpin RNA spacer region contains an intron, particularly the rolA gene intron (Pandolfini et al., 2003, BioMedCentral (BMC) Biotechnology 3:7 (www.biomedcentral.com/1472-6750/3/7)), the dual orientation introns from pHellsgate 11 or 12 (see WO 02/059294 and SEQ ID NO: 25 and 15 therein) or the pdk intron (Flaveria trinervia pyruvate orthophosphate dikinase intron 2; see WO99/53050).

Included in this definition are “siRNAs” or small interfering (double-stranded) RNA molecules of 16-30 bp, 19-28 bp, or 21-26 bp, e.g., such as the RNA forms that can be created by RNAseIII or dicer activity from longer dsRNA. siRNAs as used herein include any double-stranded RNA of 19 to 26, or 21 to 24 basepairs that can interfere with gene expression when present in a cell wherein such gene is expressed. siRNA can be synthetically made, expressed and secreted directly from a transformed cell or can be generated from a longer dsRNA by enzymatic activity. These siRNAs can be blunt-ended or can have overlapping ends. Also, modified microRNAs comprising a portion of a target gene and its complementary sequence are included herein as dsRNAs.

The term “chimeric” when referring to a gene or DNA sequence is used to refer to a gene or DNA sequence comprising at least two functionally relevant DNA fragments (such as promoter, 5′UTR, coding region, 3′UTR, intron) that are not naturally associated with each other, such as a fusion of functionally relevant DNA fragments from different sources to form an expressible chimeric gene expressing a dsRNA targeting a H. halys or other sap-feeding insect gene.

Sequences or parts of sequences which have “high sequence identity”, as used herein, refers to the number of positions with identical nucleotides divided by the number of nucleotides in the shorter of the sequences, being higher than 95%, higher than 96%, higher than 97%, higher than 98%, higher than 99%, or between 96% and 100%. A target gene, or at least a part thereof, as used herein, preferably has high sequence identity to the dsRNA of the invention in order for efficient gene silencing to take place in the target pest. Identity in sequence of the dsRNA or siRNA with a part of the target gene RNA is included in the current invention but is not necessary.

For the purpose of this invention, the “sequence identity” of two related nucleotide or amino acid sequences, expressed as a percentage, refers to the number of positions in the two optimally aligned sequences which have identical residues (×100) divided by the number of positions compared. A gap, i.e., a position in an alignment where a residue is present in one sequence but not in the other is regarded as a position with non-identical residues. The alignment of the two sequences is performed by the Needleman and Wunsch algorithm (Needleman and Wunsch, J Mol Biol, (1970) 48:3, 443-53). A computer-assisted sequence alignment can be conveniently performed using a standard software program such as GAP, which is part of the Wisconsin Package Version 10.1 (Genetics Computer Group, Madison, Wis., USA) using the default scoring matrix with a gap creation penalty of 50 and a gap extension penalty of 3.

For the purpose of the invention, the “complement of a nucleotide sequence X” is the nucleotide sequence which would be capable of forming a double-stranded DNA or RNA molecule with the represented nucleotide sequence, and which can be derived from the represented nucleotide sequence by replacing the nucleotides by their complementary nucleotide according to Chargaff's rules (A< >T; G< >C; A< >U) and reading in the 5′ to 3′ direction, i.e., in opposite direction of the represented nucleotide sequence.

A dsRNA “targeting” a gene, mRNA or protein, as used herein, refers to a dsRNA that is designed to be identical to, or have high sequence identity to, one or more mRNAs endogenous to the target organism (the target genes), and as such is designed to silence such gene upon application to such insect. One dsRNA can target one or several homologous target genes in one insect or one or several homologous target genes in different insects which can feed on the same host plant. One of skill in the art will recognize that multiple currently-known genes, as well as other currently unknown or uncharacterized genes can be targeted by applying the teachings herein.

“Insecticidal activity” of a dsRNA, as used herein, refers to the capacity to obtain mortality in insects when such dsRNA is fed to insects, which mortality is significantly higher than a negative control (using a non-insect dsRNA or buffer).

“Insect-control” using a dsRNA, as used herein, refers to the capacity to inhibit the insect development, fertility, inhibition of pheromone production, or growth in such a manner that the insect population provides less damage to a plant, produces fewer offspring, are less fit or are more susceptible to predator attack, or that insects are even deterred from feeding on such plant.

As used herein, the term “LacZ dsRNA” refers to a control dsRNA construct targeting a LacZ sequence. The LacZ protein (lacZ) is commonly used as a reporter gene in prokaryotic systems.

The term “corresponds to” as used herein means a polynucleotide sequence homologous to all or a portion of a reference polynucleotide sequence, or a polypeptide sequence that is identical to a reference polypeptide sequence. In contradistinction, the term “complementary to” is used herein to mean that the complementary sequence is homologous to all or a portion of a reference polynucleotide sequence. For example, the nucleotide sequence “TATAC” corresponds to a reference sequence “TATAC” and is complementary to a reference sequence “GTATA”. An “RNA form” of a DNA sequence, as used herein is the RNA sequence of said DNA, so the same sequence but wherein the T nucleotide is replaced by a U nucleotide.

An “effective amount” is an amount sufficient to effect desired beneficial or deleterious results. An effective amount can be administered in one or more administrations. In terms of treatment, an “effective amount” is that amount sufficient to make the target pest non-functional by causing an adverse effect on that pest, including (but not limited to) physiological damage to the pest; inhibition or modulation of pest growth; inhibition or modulation of pest reproduction; or death of the pest. In one embodiment of the invention, a dsRNA containing solution is fed to a target insect wherein critical developmental and/or reproductive functions of said insect are disrupted as a result of ingestion.

The term “plant sap-feeding organism”, “phloem-sap-feeding organism”, “xylem-sap-feeding organism”, or any grammatical variant thereof refers to any organism—typically an insect—that feeds on the sap, phloem, xylem, or two or more of these of plants. Such feeding can include penetration and sucking, piercing and sucking, scratching and sucking, or any other methods of access to the sap of a plant.

General Overview

Double-stranded RNA (dsRNA) mediated gene silencing, also known as RNA interference (RNAi), is a breakthrough technology for functional genomic studies that has potential as a tool for management of insect pests. Since the inception of RNAi numerous studies have documented successful introduction of synthetic dsRNA or siRNA into the organism that triggers a highly efficient gene silencing through degradation of endogenous RNA homologous to the presented dsRNA/siRNA. One focus of the present invention is providing for RNAi-mediated control of sap-feeding insects, including, but not limited to, the brown marmorated stink bug (BMSB, Halyomorpha halys), the pea aphid (Acyrthosiphon pisum) and the harlequin bug (Murgantia histrionica).

The BMSB, a hemipteran insect, is an invasive agricultural pest in North America. The significance of its spread has affected both the rural and urban areas especially the agricultural and specialty crops. RNAi technology can serve as a viable tool for control and management of this voracious pest, however, the major challenge to utilizing RNAi approaches delivery of effective dsRNA to the insect. Mechanical microinjection of dsRNA(s) and soaking in liquid containing dsRNA(s) are both methods that have been successfully utilized for dsRNA delivery and have been documented to elicit an effective RNAi response in laboratory studies of RNAi in insects. These techniques, however, are impracticable in an agricultural setting. Another approach has been to create transgenic plants expressing dsRNA species targeting insect pests important to that particular plant (see, e.g., WO2001037654). Creating transgenic plants on which such pests could feed is a time-consuming, economically infeasible, or, often, technically impracticable approach. Additionally, there is substantial consumer and regulatory resistance to such approaches. To be relevant for agricultural pest control, delivery of dsRNA to insect pests should be economical, efficient and advantageous for the agriculture community. dsRNA delivered through ingestion of its solution directly (Baum et al., supra), by feeding bacteria expressing dsRNA (Timmons and Fire, Nature, (1998) 395:854), or via a dsRNA-containing diet are possible strategies for inducing RNAi as an agricultural pest control methodology. Herein disclosed are compositions and methods for effective delivery of insect-specific dsRNAs orally by feeding through quick, inexpensive, and technically straight-forward “dsRNA traps”. With this state of the art delivery method, RNAi can be readily applied to many insect pests as an effective molecular biopesticide.

Double-Stranded RNA and RNA Interference

Since its inception, RNAi has proved to be a potent tool to study gene function and regulation. The advent of bioinformatics coupled with next-generation high throughput sequencing has unveiled an array of transcriptomic data available for a wide range of species at different stages of development and tissues. To attain an effective RNAi response in the biocontrol of pests, an accurate and precise mode of dsRNA delivery, efficient uptake and dsRNA stability are of utmost consideration.

Presented herein are exemplary species of dsRNA targeting several sap-feeding insects, however, one of skill in the art will recognize that the methodologies detailed herein can be used for a wide array of different individual dsRNA species. Such dsRNA species can target a single gene, target multiple homologous genes, or target multiple un-related genes (e.g., using a chimeric dsRNA). The compositions and methods of the present invention can also utilize multiple different dsRNA species. Such dsRNA species can target different portions of a single gene, target multiple homologous genes, target multiple un-related genes, and target multiple different insects.

Preferably, the dsRNAs to be used in this invention target at least one insect pest gene portion of at least 19 consecutive nucleotides occurring in identical sequence or with high sequence identity in the one or more target insects. In preferred embodiments of this invention, such dsRNAs do not silence genes of a plant host, or of other non-target animals, such as beneficial insects (e.g., pollinators), insect predators or animals such as reptiles, amphibians, birds, or mammals. Levels of homology between sequences of interest can be analyzed in available databases, e.g., by a BLAST search (see also www.ncbi.nlm.nih.gov/BLAST) or by hybridization with existing DNA libraries of representative non-target organisms. In one embodiment of this invention, the dsRNA or siRNA of the invention corresponds to an exon in a target gene.

As used herein, nucleotide sequences of RNA molecules can be identified by reference to DNA nucleotide sequences of the sequence listing. However, the person skilled in the art will understand whether RNA or DNA is meant depending on the context. Furthermore, the nucleotide sequence is identical between the types of polynucleotides except that the T-base is replaced by uracil (U) in RNA molecules.

In some embodiments, the length of the first (e.g., sense) and second (e.g., antisense) nucleotide sequences of the dsRNA molecules of the invention can vary from about 10 nucleotides (nt) up to a length equaling the length in nucleotides of the transcript of the target gene. The length of the first or second nucleotide sequence of the dsRNA of the invention can be at least 15 nt, or at least about 20 nt, or at least about 50 nt, or at least about 100 nt, or at least about 150 nt, or at least about 200 nt, or at least about 400 nt, or at least about 500 nt. If not all nucleotides in a target gene sequence are known, it is preferred to use such portion for which the sequence is known and which meets other beneficial requirements of the invention.

It will be appreciated that the longer the total length of the first (sense) nucleotide sequence in the dsRNA of the invention is, the less stringent the requirements for sequence identity between the total sense nucleotide sequence and the corresponding sequence in the target gene becomes. The total first nucleotide sequence can have a sequence identity of at least about 75% with the corresponding target sequence, but higher sequence identity can also be used such as at least about 80%, at least about 85%, at least about 90%, at least about 95%, about 100%. The first nucleotide sequence can also be identical to the corresponding part of the target gene. However, it is advised that the first nucleotide sequence includes a sequence of 19 or 20, or about 19 or about 20 consecutive nucleotides, or even of about 50 consecutive nucleotides, or about consecutive 100 nucleotides, or about 150 consecutive nucleotides with only one mismatch, preferably with 100% sequence identity, to the corresponding part of the target gene. For calculating the sequence identity and designing the corresponding first nucleotide sequence, the number of gaps should be minimized, particularly for the shorter sense sequences.

The length of the second (antisense) nucleotide sequence in the dsRNA of the invention is largely determined by the length of the first (sense) nucleotide sequence, and may correspond to the length of the latter sequence. However, it is possible to use an antisense sequence that differs in length by about 10% without any difficulties. Similarly, the nucleotide sequence of the antisense region is largely determined by the nucleotide sequence of the sense region, and may be identical to the complement of the nucleotide sequence of the sense region. Particularly with longer antisense regions, it is however possible to use antisense sequences with lower sequence identity to the complement of the sense nucleotide sequence, such as at least about 75% sequence identity, or least about 80%, or at least about 85%, more particularly with at least about 90% sequence identity, or at least about 95% sequence to the complement of the sense nucleotide sequence. Nevertheless, it is advised that the antisense nucleotide sequence always includes a sequence of 19 or 20, about 19 or about 20 consecutive nucleotides, although longer stretches of consecutive nucleotides such as about 50 nucleotide, or about 100 nucleotides, or about 150 nucleotides with no more than one mismatch, preferably with 100% sequence identity, to the complement of a corresponding part of the sense nucleotide sequence can also be used. Again, the number of gaps should be minimized, particularly for the shorter (19 to 50 nucleotides) antisense sequences.

In one embodiment of the invention, a dsRNA molecule may further comprise one or more regions having at least 94%, at least 95%, at least 96%, at least 97%, at least 98%, or at least 99%, sequence identity to regions of at least 19 consecutive nucleotides from the sense nucleotide sequence of the target gene, different from the at least 19 consecutive nucleotides as defined in the first region, and one or more regions having at least 94%, at least 95%, at least 96%, at least 97%, at least 98%, or at least 99%, sequence identity to at least 19 consecutive nucleotides from the complement of the sense nucleotide sequence of the target gene, different from the at least 19 consecutive nucleotides as defined in the second region, wherein these additional regions can base-pair amongst themselves.

dsRNA-Containing Plant Structures (“dsRNA Traps”)

Described herein are novel compositions containing dsRNA(s) and methods of using them that target one or more chosen pest insects. The invention takes advantage of the vascular and/or osmotic flow of materials through living plant tissue to distribute a non-naturally-occurring dsRNA species throughout an intact and living plant material (e.g., a fruit, vegetable, leaf, stem, etc.) on which a sap-feeding insect can feed. Typically, the living plant material is at least partially soaked in an aqueous solution containing the one or more dsRNA species to be loaded into it for a sufficient time to allow for uptake of the dsRNA(s). Such a procedure can involve removal of a portion of the living plant material to provide access to the vascular structures. The mechanism(s) by which the living plant material takes up and distributes the dsRNA throughout its tissues is not relevant, as long as the plant material can perform these actions.

In preferred embodiments, the plant material is an attractive food source for the one or more insect pests targeted. Thus, in practicing the present invention, a variety of structures from various plants can be utilized including, but not limited to, leaves, fruits, stems and vegetables. In preferred embodiments, the plant material is capable of being fed upon by a sap-feeding insect. By way of example only, and not intended to limit the specific sources of plant materials, certain embodiments of the present invention can include dsRNA(s) taken up and distributed through vegetables (e.g., cucumbers, green beans, snow peas, sugar snap peas, etc.), fruits (e.g., strawberries, apples, cherries, etc.), stems (e.g., tomato, cantaloupe, etc.), leaves (e.g., collard greens, spinach, kale, lettuce, etc.). One of skill in the art will recognize that the particular plant structure to serve as a source of dsRNA ingestion by a target insect pest can be chosen on the basis of multiple factors, such as the ability of the plant material to uptake the dsRNA(s), the ability of the target insect pest(s) to feed on the plant structure and the attractiveness of the plant material to the target insect pest(s).

By varying the concentration of dsRNA in the solution in which plant material is soaked, various concentrations throughout the plant structure can be achieved. Additionally, the plant material can be trimmed to desired lengths to achieve a known concentration over a given length. Thus, in some embodiments of the invention, a particular concentration can be achieved per unit length of the plant material. Such concentrations include concentrations anywhere from 0.01 μg/inch to 10 μg/inch, for example 0.01 μg/inch, 0.02 μg/inch 0.03 μg/inch, 0.04 μg/inch, 0.05 μg/inch, 0.06 μg/inch, 0.07 μg/inch, 0.08 μg/inch, 0.09 μg/inch, 0.1 μg/inch, 0.2 μg/inch, 0.3 μg/inch, 0.4 μg/inch, 0.5 μg/inch, 0.6 μg/inch, 0.7 μg/inch, 0.8 μg/inch, 0.9 μg/inch, 1.0 μg/inch, 1.1 μg/inch, 1.2 μg/inch, 1.3 μg/inch, 1.4 μg/inch, 1.5 μg/inch, 1.6 μg/inch, 1.7 μg/inch, 1.8 μg/inch, 1.9 μg/inch, 2.0 μg/inch, 2.1 μg/inch, 2.2 μg/inch, 2.3 μg/inch, 2.4 μg/inch, 2.5 μg/inch, 2.6 μg/inch, 2.7 μg/inch, 2.8 μg/inch, 2.9 μg/inch, 3.0 μg/inch, 3.1 μg/inch, 3.2 μg/inch, 3.3 μg/inch, 3.4 μg/inch, 3.5 μg/inch, 3.6 μg/inch, 3.7 μg/inch, 3.8 μg/inch, 3.9 μg/inch, 4.0 μg/inch, 4.1 μg/inch, 4.2 μg/inch, 4.3 μg/inch, 4.4 μg/inch, 4.5 μg/inch, 4.6 μg/inch, 4.7 μg/inch, 4.8 μg/inch, 4.9 μg/inch, 5.0 μg/inch, 5.1 μg/inch, 5.2 μg/inch, 5.3 μg/inch, 5.4 μg/inch, 5.5 μg/inch, 5.6 μg/inch, 5.7 μg/inch, 5.8 μg/inch, 5.9 μg/inch, 6.0 μg/inch, 6.1 μg/inch, 6.2 μg/inch, 6.3 μg/inch, 6.4 μg/inch, 6.5 μg/inch, 6.6 μg/inch, 6.7 μg/inch, 6.8 μg/inch, 6.9 μg/inch, 7.0 μg/inch, 7.1 μg/inch, 7.2 μg/inch, 7.3 μg/inch, 7.4 μg/inch, 7.5 μg/inch, 7.6 μg/inch, 7.7 μg/inch, 7.8 μg/inch, 7.9 μg/inch, 8.0 μg/inch, 8.1 μg/inch, 8.2 μg/inch, 8.3 μg/inch, 8.4 μg/inch, 8.5 μg/inch, 8.6 μg/inch, 8.7 μg/inch, 8.8 μg/inch, 8.9 μg/inch, 9.0 μg/inch, 9.1 μg/inch, 9.2 μg/inch, 9.3 μg/inch, 9.4 μg/inch, 9.5 μg/inch, 9.6 μg/inch, 9.7 μg/inch, 9.8 μg/inch, 9.9 μg/inch, 10.0 μg/inch, or more. One of skill in the art will recognize that, although these values are provided in μg/inch values, any concentrations within these ranges expressed in other concentration per unit length are contemplated herein. The ranges provided also encompass all incremental concentrations between the specifically stated points.

In preferred embodiments, the present invention provides a composition having at least one inhibitory nucleic acid specific for an mRNA, fragment thereof, or homologue thereof present in a target insect pest. Typically, dsRNA(s) of the present invention are provided to a target insect pest in an amount sufficient to inhibit production of the targeted polypeptide encoded by one or more of the full-length genes targeted by selected dsRNA(s) or homologues and alleles thereof. For example when a target insect is feeding on dsRNA-laden plant material (e.g., vegetable or fruit) containing an inhibitory nucleic acid, the insect ingests a sufficient level of dsRNA to result in a phenotypic effect. In particular embodiments, a combination of two or more dsRNAs are combined in a single plant material. In embodiments where two or more dsRNAs are combined in a single plant material the dsRNAs can target different genes or different portions of the same gene from the same or different pest targets. Thus, in one embodiment, a single plant material can be used to deliver multiple, different dsRNA species targeting the production of one or more proteins from one or more pests. Where two or more dsRNAs are taken up and distributed throughout the vascular tissue by a plant material, the dsRNAs can be provided to the plant material in a single solution, or in multiple, sequentially-applied solutions.

In addition to an inhibitory nucleic acid, a dsRNA-containing plant material of the present invention can also comprise one or more chemoattractants, phagostimulants, visual attractants, insecticides, pheromones, fungicides, or combinations thereof. Such additional components are well known in the art and are readily chosen to complement compositions of the present invention, but are not specifically integral to the present invention. These additional components can be formulated to be coated on a plant, plant part, leaf, fruit, vegetable, stem or other plant structure. In certain aspects the additional component(s) are combined with one or more excipients, buffering agents, carriers, etc. Excipients, buffering agents, and carriers are also well known in the art.

Where additional components are applied in a coating, the coating can be formulated as a spray or dip so that the additional non-dsRNA components acids remain on the exterior of the plant material. For example, a leaf having a dsRNA distributed through at least part of its vascular system can be coated with a composition comprising one or more chemoattractants, phagostimulants, visual attractants, insecticides, pheromones, fungicides, or combinations thereof. Alternately, the additional component can be mixed with an aqueous solution containing the dsRNA(s) to be taken up and distributed via vascular action of the plant material, or osmosis through the plant material, thus distributing the dsRNA(s) and the additional component(s) throughout at least part of the plant material.

Having described the invention in general, below are examples illustrating the generation and efficacy of the invention. Neither the examples, nor the general description above should be construed as limiting the scope of the invention.

### Examples

Artificial Diets:

Four different artificial diets were prepared for the experiments detailed herein. First was the artificial gypsy moth diet. This diet was prepared by combining wheat germ 120 g, USDA vitamin Mix 10 g, casein 25 g, Wesson salts 8 g, sorbic acid 2.5 g, methyl paraben 1 g, agar 15 g and water 825 ml. The ingredients were added to a high-speed blender in warm water, blended and poured into 96 well plates. Additionally, were the artificial BMSB diets (2% or 8%). This diet was prepared by combining agar 10 g (for 2% agar diet) or 40 g (for 8% agar diet), applesauce 113 g (Santacruz organic), organic apple juice 50 ml, water to 500 ml. Finally, was the artificial BMSB diet with green bean puree. This diet was prepared by combining wheat germ 60 g, USDA vitamin Mix 10 g, Wesson salts 8 g, Sorbic Acid 2.5 g, methyl paraben 1 g, Agar 15 g, cellulose 50 g, organic green beans (boiled and pureed) 200 g, dextrose 75 g, sucrose 25 g, water to make up to 1 L.

These diets were poured into 96-well polypropylene plates, frozen overnight (−20° C.), and dried in a Virtis Advantage Freeze Drier (The Virtis, Gardiner, N.Y.) for freeze drying. The Frozen diets in 96-well plates were placed in the pre-frozen shelves −45° C. and held for 20 min. The diets were further dried in the following steps under vacuum at 15 mTorr: −40° C. for 600 min, −30° C. for 420 min, −20° C. for 300 min, −10° C. for 300 min, 0° C. for 60 min, 10° C. for 60 min, 20° C. for 120 min, 30° C. for 120 min, and 40° C. for 120 min. The initial four steps are the primary drying phase and the last six steps are essential for secondary drying. The secondary drying steps are necessary to ensure the ability of the diet to absorb the treatment solution. The vacuum is released following completion of the freeze-drying program; the 96-well plates were removed and inverted to remove the diet pellets. These pellets were then placed in sterile plastic bags, and stored at 4° C. prior to use.

Vegetable Diets:

Two different vegetable diets were used. For the Green Bean diet, green beans were washed with 0.2% sodium hypochlorite solution (J. T. Baker) for five minutes and later washed 3 times with ddH₂O. The beans were trimmed from the calyx end to a total length of 3 inches. These beans were used as controls. For delivery of dsRNA treatment through green beans, green beans were washed and trimmed as mentioned above. Next the beans were immersed in a cap less 2 ml microcentrifuge tube containing a 300 ml solution containing 1:10 dilution of green food coloring or 5 μg or 10 μg of dsRNA in RNase DNase free water. Lean green beans were selected for this diet to ensure the beans fit in the 2 ml microcentrifuge tubes. To prevent any evaporation of the solution or animals entering the solution, the microcentrifuge tubes containing the beans were sealed with parafilm. These tubes were kept at room temperature for 3 hours allowing for the solution to rise to the style of the green bean through capillary action. The tubes were further placed in a small box to keep them upright and enclosed in magenta jars (Sigma).

For the Collard Green diet (utilized as a dsRNA delivery system for HB (M. histrionica)), young collard green (Brassica oleracea var. viridis) leaves measuring about 3-4 inches in length were snipped at the petiole, washed and trimmed as mentioned above. Next the leaves were immersed at the petiole end in a cap less 2 ml microcentrifuge tube containing a 300 ml solution containing either RNase DNase free water or 1:10 dilution of green food coloring in RNase DNase free water. To prevent any evaporation of the solution or animals entering the solution, the microcentrifuge tubes containing the leaves were then sealed with parafilm. These tubes were stored at room temperature for 3 hours allowing for the solution to rise to the leaf surface. The tubes were further placed in a small box to keep them upright and enclosed in magenta jars (Sigma).

BMSB is a sap (phloem) feeder causing damage by piercing and sucking from the vascular tissues of the fruit and vegetables. The selection of green beans as the vegetable for delivery rested on several factors, including the ready availability, low cost, preference of BMSB for this particular plant material and ability of BMSB to grow and prosper on a green bean diet. We also tested several artificial diets alongside the natural diets to select the green beans for delivering dsRNA to BMSB. A similar experiment was attempted by rehydrating freeze-dried organic apples or injecting organic raisins with a 10% sugar solution but yielded limited success (data not shown). As detailed below, this vegetable delivery system allows both for the uptake of in vitro synthesized dsRNA and also for the effective delivery of inhibitory dsRNA species (as demonstrated below with the successful depletion of target genes in the tissues of BMSB).

Vegetable-Mediated Delivery of Treatment:

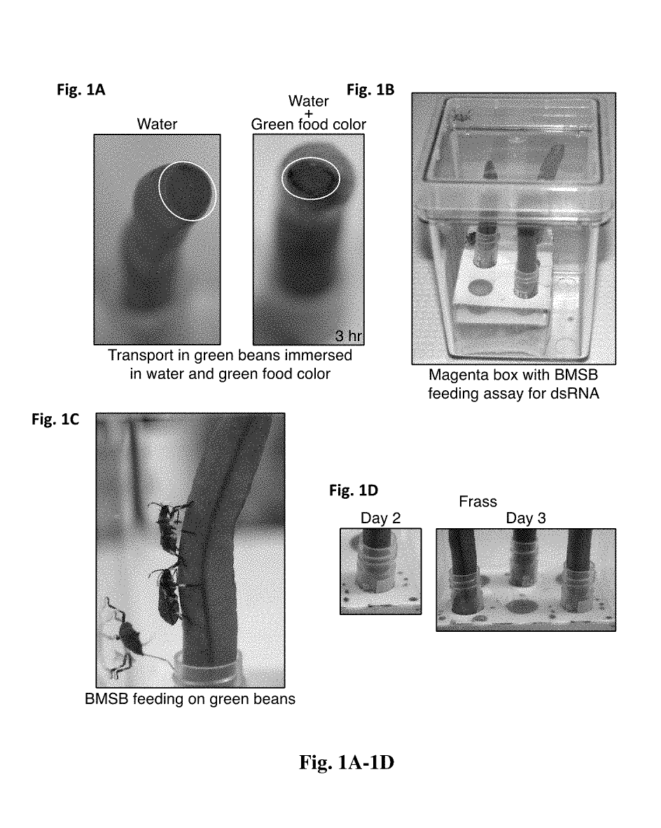

In hemipteran insects the most common experimental mode of dsRNA delivery has been using mechanical microinjections to deliver dsRNA directly to the haemolymph (Jaubert-Possamai et al., supra). This approach, however, is impracticable for insect pest control purposes. Therefore, we sought to develop an oral delivery approach for biocontrol using RNAi in the invasive insect pest BMSB and tested a vegetable delivery method. Lean organic green beans (Phaseolus vulgaris) were selected as a medium for delivery of dsRNA, or other treatments, to the animal. BMSB feeds on this cultivar crop by piercing into the vascular tissue using their needlelike stylets. The BMSB feeds by alternate salivation and ingestion with slow movement of stylets in lacerate-and-flush feeding method causing considerable damage to the cultivar crops (Peiffer and Felton, PLoS ONE (2014) 9:e88483). We used this feeding technique to our benefit by testing the delivery of green food color compared to water. A solution of green food color was mixed at 1:10 ratio with water to imitate dsRNA. Slender green beans were trimmed from the calyx end for a total length of 3 inches. These beans were inverted and immersed into either the food color solution or water in a 2 ml microcentrifuge tube. Due to either the flow of phloem or capillary action the solution was allowed to reach the style of the bean through the vascular tissue. This is indicated by the green coloration of the peripheral vascular tissue at the style (FIG. 1A). A total of three beans were placed in the magenta vessels and a group of 3 animals were treated per vessel (FIG. 1B).

H. halys (BMSB) insects were reared at USDA-ARS in the Beltsville Agricultural Research Center, Beltsville, Md. (Khrimian A 2014). This colony was established in 2007 from adults collected in Allentown, Pa., and supplemented annually until 2012 with several animals collected at Beltsville, Md. Insects were reared in ventilated plastic cylinders (21621 cm OD) on a diet of organic green beans, shelled sunflower and buckwheat seeds (2:1, w/w), and distilled water supplied in cotton-stopped shell vials. Eggs were collected weekly, hatched in plastic Petri dishes with a water vial. Once the animals molted to second-instars, nymphs were transferred to larger rearing cages till adults. Adults, males and females were separated 1 to 2 days post emergence, and subsequently maintained in different containers. Insects were maintained in Thermo Forma chambers (Thermo Fisher Scientific) at 25° C. and 72% relative humidity, under a 16 L:8D photoperiod.

Early fourth instar nymphs were selected primarily from the same egg mass and starved for 24 hrs before resuming feeding. The animals were treated in groups of three per magenta vessel containing three green beans, or three green beans with green food color or dsRNA, or 4 freeze dried pellets rehydrated with 0.5 ml of 10% sugar solution. The diets were replenished as per experimental requirements. The animals fed on the upright green beans by inserting their stylets into the vascular tissues (FIG. 1C). If the animal fed on the green food color then the green frass subsequently produced was indicative of oral delivery of treatment (passed through the gut before excreted). Green frass was observed on day 2 of feeding, visualized as green dots, which further increased in content on the third day (FIG. 1D).

Selection of a Suitable Diet:

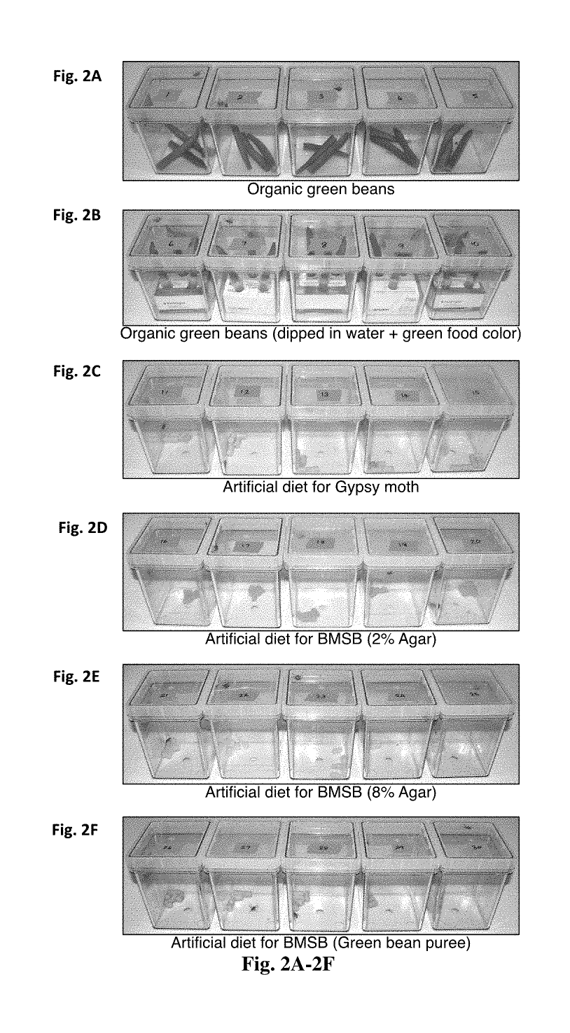

Next we tested the feasibility of an optimal artificial diet compared to vegetable diet for delivery of dsRNA. Four different artificial diets were prepared comprising of various ingredients (FIGS. 2C-F). These diets primarily contained ingredients that are favorable to BMSB consisting of applesauce and apple juice (FIGS. 2D & 2E) or green bean puree (FIG. 2F) (Bell et al. 1981, in Doane and McManus, “The Gypsy Moth: Research Toward Integrated Pest Management”, 1981, pg. 599, Technical Bulletin, USDA). Artificial diets were prepared and freeze dried so the treatment can be delivered using a solution that can rehydrate the pellets without disintegrating the diet. Organic green beans were fed as control while green beans immersed in water were fed as a new delivery method (FIGS. 2A & B).

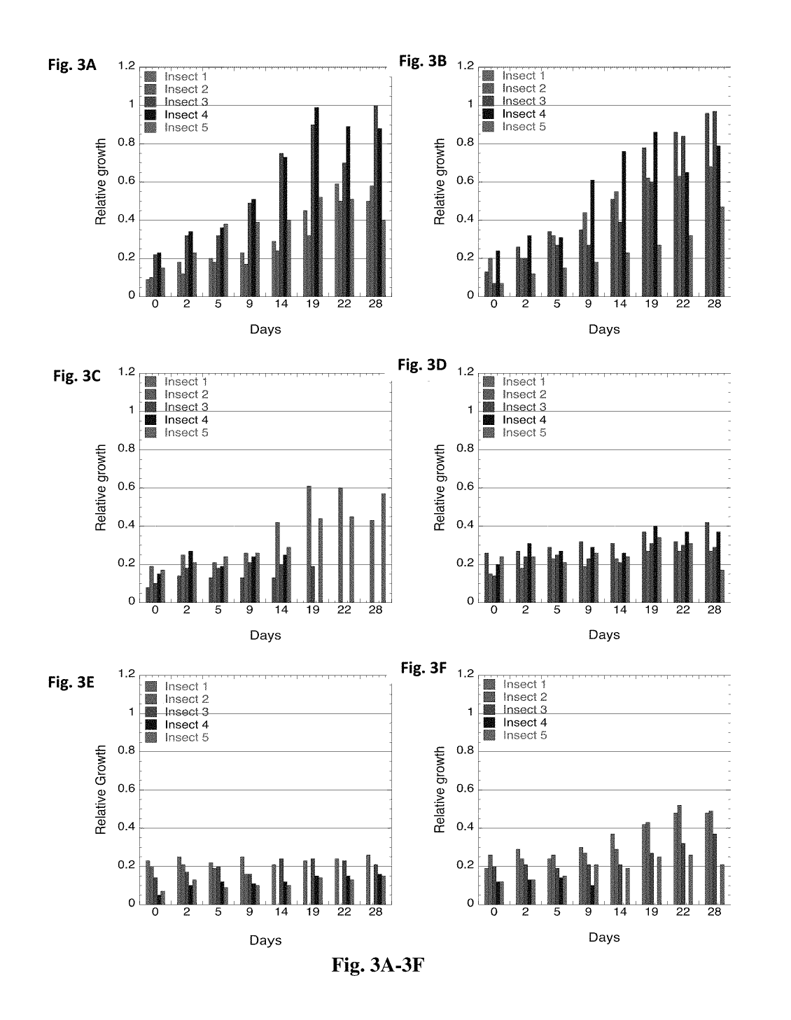

The animals were allowed to feed on these diets for a period of 4 weeks and monitored for any physiological changes. The diets were changed every 3 days and replenished with new diets. The animals were observed as individuals and a record of their physiological condition was also monitored. The animals feeding on artificial diet showed remarkable survival. Although the animals had a similar body mass until day 2 of the feeding when compared to the control, by day 22 the body mass decreased by 40% with 40% survival (compare FIG. 3A to FIG. 3C). Body mass of insects fed on the other diets consisting of applesauce or green bean puree also indicated a 60% decrease with a significantly lower mortality as shown in FIG. 3C and compared to the control. Upon comparing animals fed on green beans immersed in water to the control, they displayed no mortality and stable increase of body mass (compare FIG. 3A to FIG. 3B) indicating the green bean diet was a better method for delivery of dsRNA.

In Vitro Synthesis of Double Stranded RNA:

Genes specific to BMSB were selected by examining the transcriptomic profiles (Sparks et al., PLoS ONE (2014) 9:e111646), and regions of interest for each gene selected that varied between 200 to 500 base pairs. PCR products were then generated by polymerase chain reaction (PCR) by amplifying genomic DNA using specific oligonucleotides and purified using a PCR purification kit (Qiagen). This PCR amplified region was then used as template generate dsRNA required for RNAi in BMSB. The primers used for PCR contained the T7 promoter sequence (5′-GAA TTA ATA CGA CTC ACT ATA GGG AGA-3′). LacZ, a gene that encodes β-galactosidase, was amplified from the E. coli genomic DNA and served as a negative control (mock) for RNAi (all primers used are listed in Table 1).

TABLE 1
PCR Primers.

| | | | SEQ |
|---|---|---|---|
| Gene | Direction | Sequence (5′-3′) | ID NO. |
| Vitellogenin | Forward | CAATTTGATCCACCGA | 1 |
| (BMSB) | | CTGTT | |
| Vitellogenin | Reverse | CCGCATGAATCTTACT | 2 |
| (BMSB) | | CTGGA | |
| Juvenile | Forward | GGATGCTTATGAATAA | 3 |
| hormone | | TCCAG | |
| (BMSB) | | | |
| Juvenile | Reverse | GTATAGGATTGCCATT | 4 |
| hormone | | TTGG | |
| (BMSB) | | | |
| Vitellogenin- | Forward | GAATTAATACGACTCA | 5 |
| T7 (BMSB) | | CTATAGGGAGACCAAA | |
| | | GTTGGAAGGGAATGA | |
| Vitellogenin- | Reverse | GAATTAATACGACTCA | 6 |
| T7 (BMSB) | | CTATAGGGAGACCGCA | |
| | | TGAATCTTACTCTGGA | |
| Juvenile | Forward | GAATTAATACGACTCA | 7 |
| hormone-T7 | | CTATAGGGAGAGGATG | |
| (BMSB) | | CTTATGAATAATCCAG | |
| Juvenile | Reverse | GAATTAATACGACTCA | 8 |
| hormone-T7 | | CTATAGGGAGAGTATA | |
| (BMSB) | | GGATTGCCATTTTGG | |
| LacZ-T7 | Forward | GAATTAATACGACTCA | 9 |
| | | CTATAGGGAGATGAAA | |
| | | GCTGGCTACAGGA | |
| LacZ-T7 | Reverse | GAATTAATACGACTCA | 10 |
| | | CTATAGGGAGAGCAGG | |
| | | CTTCTGCTTCAAT | |

The PCR-amplified DNA was purified using a PCR purification kit (Qiagen). In vitro transcription to yield dsRNA was performed by combining 250 mM HEPES phH 7.5, 32 mM magnesium chloride, 10 mM Dithiothreitol (DTI), 2 mM spermidine, 25 mM each of rNTPs, 0.25 units of SUPERase In RNase inhibitor (Life Technologies), and 0.5 μg PCR amplified DNA in a final volume of 20 μl were incubated at 37° C. for 5 min. After 5 min, 1 μg T7 RNA polymerase was added to the reaction and further incubated at 37° C. overnight.

The reactions were then centrifuged for 2 min at 13,000 rpm to pellet the magnesium pyrophosphate. The supernatant was transferred was treated with 2 units of RQ1 DNase followed by incubation at 37° C. for 30 min. The reaction mixture was extracted with an equal volume of phenol/chloroform/isoamyl alcohol (25:24:1) and centrifuged. The aqueous layer was extracted with chloroform. One-fifth-volume ammonium acetate (5 M ammonium acetate+100 mM EDTA) and 3 volumes of chilled 100% ethanol were added to the resulting aqueous layer. After incubating on ice for 10 min, the dsRNA was precipitated, washed with 75% ethanol, resuspended in nuclease free water and stored in the freezer for use. dsRNA species utilized are listed in Table 2.

TABLE 2
dsRNA species for RNAi analysis

| | SEQ | |
|---|---|---|
| | ID | |
| Source | NO. | Sense Strand Sequence |
| Vitellogenin- | 11 | GTTAAACTAGGTGGCTGACAAGAAAAA |
| A1-like | | GACTCGCGACTAGTTCATTCTTAAGCT |
| (BMSB) | | AGACACCGCGGAGTAAGTAAACTCCAA |
| | | CACCTCCTTCTATAAGAAGCGATCTCG |
| | | ACTAACTACTTGAGAGTGATCTCCTAC |
| | | TTCTCGAACTCCTGCTTCTTCGAGGAA |
| | | CAGATTAACCCAATATGGACAAAAGGA |
| | | GTCTGCTTGATGATCTCGATCAACTAA |
| | | GCGAACTTGACGACAACCCAACTCCCC |
| | | CTCCACAACGACTAGAACAAAAAGAGA |
| | | AGTATGAAACTAAGGTAAATAAAAGAA |
| | | GTGATGACCTCCTTCTCGAACTCCTGC |
| | | TTCTACTTCTTCTGCTACTTCAACTTA |
| | | GTCTTGGAAGCGATGATAGTGGTAAAA |
| | | CACCTATAGGTATAAGTATAAGCATCG |
| | | GGTTTAATAATTTGCGGAATCCTGATA |
| | | AATACGAGTCCAGACTTGGAAACACCC |
| | | CGTGATGGTATAGGTCTCATTCTAAGT |
| | | ACGCC |
| Juvenile | 12 | GGATGCTTATGAATAATCCAGAGCTGT |
| hormone | | ATACAAATGTAAATGCATTGCAAAAAC |
| acid O- | | GCGATGCACAAGAGGTCTTGGAAGAAG |
| methyl- | | TTAAAGATCTATTACCATGGTCTATAG |
| transferase- | | GAGAAAACGTGCTAGATGTTGGCTGTG |
| like | | GACCTGGTGATCTCACATCCTCCCTTC |
| (BMSB) | | TCACTTCATATCTGGCCAATGACTATC |
| | | GAGTGGTCGGTTGCGATATTTCTGAAG |
| | | CTATGGTGAAATATGCTCAAGCAAAAT |
| | | ATGGAAACGATCAATTTTGTTTCAAAC |
| | | AGCTTGATATCAGCAATGGAAATATAT |
| | | GGATGAACTGGGAAGAGGAGATTTTTG |
| | | ATAAAGTATTTTCATTTTACTGCCTTC |
| | | ACTGGGTTAAAGATCAGATACAAGCAG |
| | | CAGAAAACATTTATAGTTTGCTGAAAG |
| | | ATGGTGGTTATTTTGTCACAATGTTCA |
| | | CTATATCTCATCCGTTTCTTATTCTAT |
| | | TTAGCAGACTTAAGGAAAACCCAAAAT |
| | | GGCAATCCTATAC |
| LacZ | 13 | TGAAAGCTGGCTACAGGAAGGCCAGAC |
| (E. coli) | | GCGAATTATTTTTGATGGCGTTAACTC |
| | | GGCGTTTCATCTGTGGTGCAACGGGCG |
| | | CTGGGTCGGTTACGGCCAGGACAGTCG |
| | | TTTGCCGTCTGAATTTGACCTGAGCGC |
| | | ATTTTTACGCGCCGGAGAAAACCGCCT |
| | | CGCGGTGATGGTGCTGCGCTGGAGTGA |
| | | CGGCAGTTATCTGGAAGATCAGGATAT |
| | | GTGGCGGATGAGCGGCATTTTCCGTGA |
| | | CGTCTCGTTGCTGCATAAACCGACTAC |
| | | ACAAATCAGCGATTTCCATGTTGCCAC |
| | | TCGCTTTAATGATGATTTCAGCCGCGC |
| | | TGTACTGGAGGCTGAAGTTCAGATGTG |
| | | CGGCGAGTTGCGTGACTACCTACGGGT |
| | | AACAGTTTCTTTATGGCAGGGTGAAAC |
| | | GCAGGTCGCCAGCGGCACCGCGCCTTT |
| | | CGGCGGTGAAATTATCGATGAGCGTGG |
| | | TGGTTATGCCGATCGCGTCACACTACG |
| | | TCTGAACGTCGAAAACCCGAAACTGTG |
| | | GAGCGCCGAAATCCCGAATCTCTATCG |
| | | TGCGGTGGTTGAACTGCACACCGCCGA |
| | | CGGCACGCTGATTGAAGCAGAAGCCTG |
| | | C |

Total RNA Isolation and cDNA Synthesis:

To measure the level of gene expression in BMSB, the whole animal was homogenized using a micro-pestle subsequent to dsRNA treatment. Total RNA was isolated from the tissue samples by soaking and homogenizing in 1 ml volume of TRIzol (Invitrogen). Reverse transcriptase PCR was used to generate cDNA, 200 ng of total RNA was incubated with a 0.5 mM deoxynucleoside triphosphate mixture, 0.65 μM each oligo(dT)₁₆ (Life Technologies), and random hexamers (Life Technologies) at 65° C. for 5 min. A cDNA synthesis mixture containing 10 mM dithiothreitol (DTT), 100 units of SUPERSCRIPT REVERSE TRANSCRIPTASE III (Life Technologies), and 2 units of SUPERase In RNase inhibitor (Life Technologies) was then added to the total RNA mixture, which was incubated at 25° C. for 5 min, 50° C. for 50 min. The reaction was terminated by incubation at 70° C. for 15 min. The resulting cDNA was then evaluated with primers listed in Table 3 for specific genes by qPCR.

TABLE 3
qPCR Primers.

| | | | SEQ |
|---|---|---|---|
| Gene | Direction | Sequence (5′-3′) | ID NO. |
| Vitellogenin | Forward | TTGATAGTTGTTTGGA | 14 |
| (BMSB) | | TTTTGAAGGT | |
| Vitellogenin | Reverse | TCTTACTTGATCAGCG | 15 |
| (BMSB) | | CTCAGAA | |
| Juvenile | Forward | AGGAAAACCCAAAATG | 16 |
| hormone | | GCAAT | |
| (BMSB) | | | |
| Juvenile | Reverse | ATGTATTCTTCTTTTG | 17 |
| hormone | | GATCTTTTCTTGAG | |
| (BMSB) | | | |
| 18S (BMSB) | Forward | ATGCCCCCGCCTGTCC | 18 |
| | | TTATT | |
| 18S (BMSB) | Reverse | TGAAAGCAGCCTGAAT | 19 |
| | | AGTGG | |

Quantitative Real-Time PCR Analysis:

Levels of transcripts expressed were measured by quantitative realtime PCR (qPCR) using SYBR green PCR master mix from SENSIMIX SYBR from Bioline. The reactions were performed on an Applied Biosystems 7500 real-time PCR system. Data were analyzed with ABI Prism sequence detection system software. All analysis was performed in the linear range of amplification. Standards were determined by serial dilution of the cDNA prepared from total RNA isolated from gut tissue of a normal animal and used as a reference standard for the quantification of cDNA produced from RNA. 18s RNA was used as an internal standard to correct for differences in RNA recovery from tissues (Sparks et al., supra). The data was plotted using KALEIDAGRAPH (Synergy software).

Mobility of In Vitro Transcribed dsRNA Through Green Bean Diet:

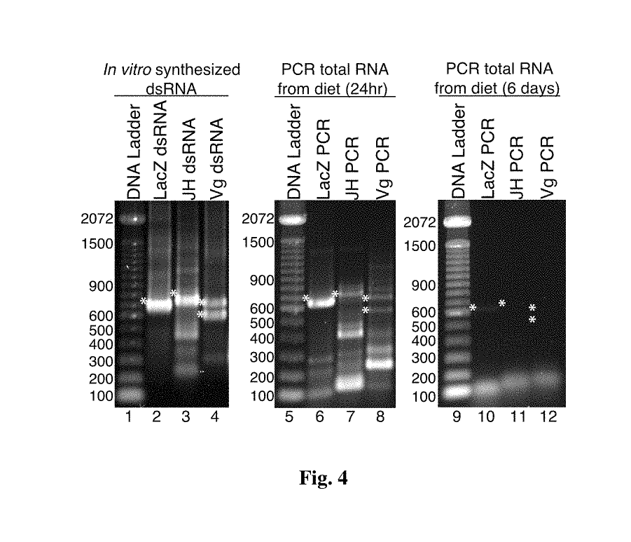

Mobility of green food color facilitated via green beans indicated that when the bean is immersed upright in a solution it could rise against gravity to the bean style. We further tested whether this phenomenon occurred for dsRNA as well as green food color and, if so, whether the nucleic acid was stable when delivered through the beans' vascular tissues. To test this in vitro synthesized dsRNA for BMSB-specific genes—JH (SEQ ID NO. 11) and vitellogenin (SEQ ID NO. 12)—were selected for RNAi analysis, while LacZ, a gene encoding β-galactosidase amplified from E. coli genomic DNA, was used as negative control (mock) (FIG. 4A). For these studies, the beans were immersed for 24 hrs in an aqueous solution containing 5 μg of dsRNA at concentrations of 1.67 μg/inch. Subsequently, 0.5 cm of the stylus region was clipped followed by excision of a 1 cm region from the bean was tested. Total RNA was isolated and analyzed by PCR using gene specific oligonucleotides (Travaglini and Loeb, Biochem., (1974) 13:3010-17; Tse and Forget, Gene, (1990) 88:293-96). Results indicated that dsRNA introduced into the beans for delivery was stable when compared to the dsRNA synthesized prior to its uptake in the bean (FIG. 4, compare lanes 2-4 to 5-7). This demonstrated that BMSB-specific dsRNA, though foreign to green beans, was successfully transported through the vascular tissues of the bean to the style serving as a vehicle for dsRNA transport.

In addition to the stability of dsRNA delivered through green bean, the persistence of dsRNA is of importance to feed sufficient levels to target pest insects. It has been previously demonstrated that dsRNA is degraded and biologically inactive in soil after a period of 36 hours indicating accumulation or persistence of dsRNA in the environment is unlikely (Dubelman et al., PLoS ONE, (2014) 9:e93155). To assess the persistence of dsRNA in the present system, a study was performed to validate its degradation in green beans. The beans were immersed in 5 μg of dsRNA for 6 days and total RNA was isolated and analyzed by PCR as mentioned above. Results implied that persistence of dsRNA after 6 days was greatly diminished for LacZ transcript as compared to 24 hr (FIG. 4 compare lanes 6 and 10). However, for the JH (SEQ ID NO. 11) and Vg (SEQ ID NO. 12) dsRNA species, PCR amplified products were undetectable as dsRNA may have degraded beyond detection (FIG. 4, compare lanes 7, 8 to 11, 12).

This study demonstrated the stability of different dsRNA species when using a vegetable-mediated delivery technique as verified by the depletion in the level of targeted gene transcripts and the amount of PCR product detected after 24 hr in the green beans. The biodegradation was measured using PCR amplification of total RNA from the vegetable, which revealed rapid degradation of JH and Vg dsRNA in the green beans. Though negligible amounts of LacZ PCR amplicons were still visible, we speculate that if the dsRNA was allowed to incubate for a longer period of time in the beans this also would have degraded. This demonstrates that the disclosed “dsRNA traps” (vegetables, fruits or other living plant structures laden with exogenously produced dsRNA species) will not result in an accumulation of the dsRNA species in the environment. We speculate that the degradation of the dsRNA is via any of the well-characterized mechanisms possessed by eukaryotes that process and degrade dsRNAs (Gantier and Williams, Cytokine Growth Factor Rev., (2007) 18:363-71).

RNAi in BMSB Using Green-Bean-Mediated dsRNA Delivery:

Next we investigated if RNAi can be successfully achieved to deplete specific genes in the invasive insect pest, BMSB through feeding on dsRNA-containing green beans. To test this system, beans were immersed in a solution of either 5 or 20 μg of in vitro synthesized dsRNA of JH (SEQ ID NO. 11) and Vg (SEQ ID NO. 12). Another set consisting of beans immersed only in water, or LacZ dsRNA (mock), was also used as controls. Three BMSB 4^{th} instar nymphs were allowed to feed on dsRNA-laden and control green beans in a magenta vessel as described above (FIG. 2) for a period of five days. The levels of gene expression were evaluated using qPCR for three biological replicates.

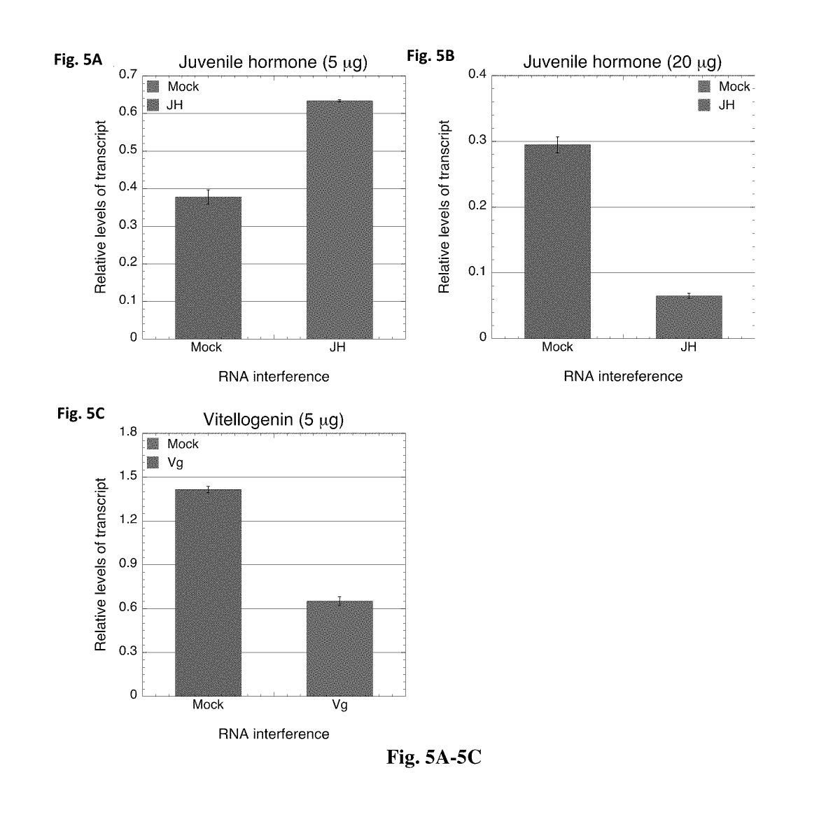

Observations revealed that when the animals were allowed to feed on beans immersed in 5 μg solution of dsRNA of JH (SEQ ID NO. 11) and Vg (SEQ ID NO. 12) only the level of Vg transcript was significantly depleted by approximately 2.2-fold (FIGS. 5A and 5C). When the animals were fed on beans immersed in a solution containing 20 μg of JH dsRNA, we observed a considerable 4.5-fold decrease in the level of JH expression (FIG. 5B). These results indicate RNAi mediated gene silencing can be accomplished using the vegetable delivery protocol. We also infer that using this delivery method the concentration of required dsRNA can be delivered in a dose dependent manner for an effective RNAi. Furthermore, these results show that our system works to deliver diverse and varied dsRNA species. These results further suggest that a single vegetable can be loaded with multiple, different dsRNA species. Thus, a single vegetable (or other dsRNA-laden plant material capable of being fed upon by a target pest insect) can be used to deliver multiple, different dsRNA species targeting one or more pests.

This study implies that RNAi could be successfully achieved using this vegetable-mediated dsRNA delivery system. This delivery system can serve as a trapping system alone, or in combination with other components intended to increase efficacy of delivery to a target insect (e.g., pheromones, chemoattractants, phagostimulants, etc.) for gene regulation in pests. As detailed below, this approach—vegetable-mediated delivery of inhibitory dsRNA species—can be applied to other hemipteran insects such as the harlequin bug and pea aphid. Thus, this approach can be utilized for a wide array of sap-feeding insects.

Vegetable Mediated Delivery in Other Hemipteran Insects:

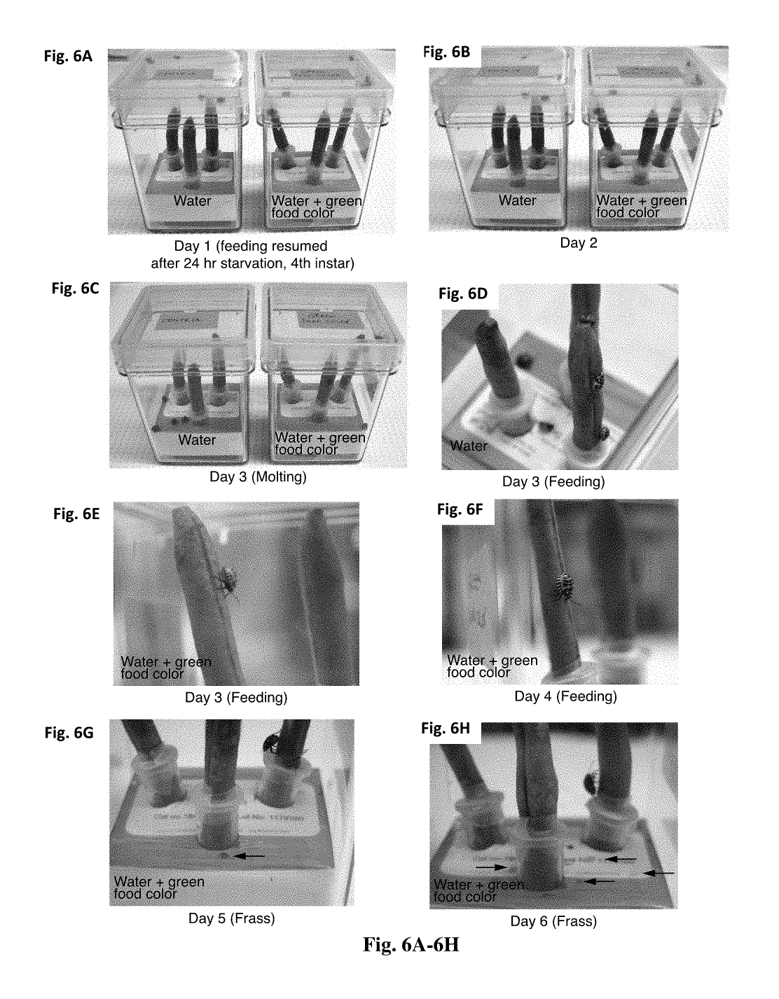

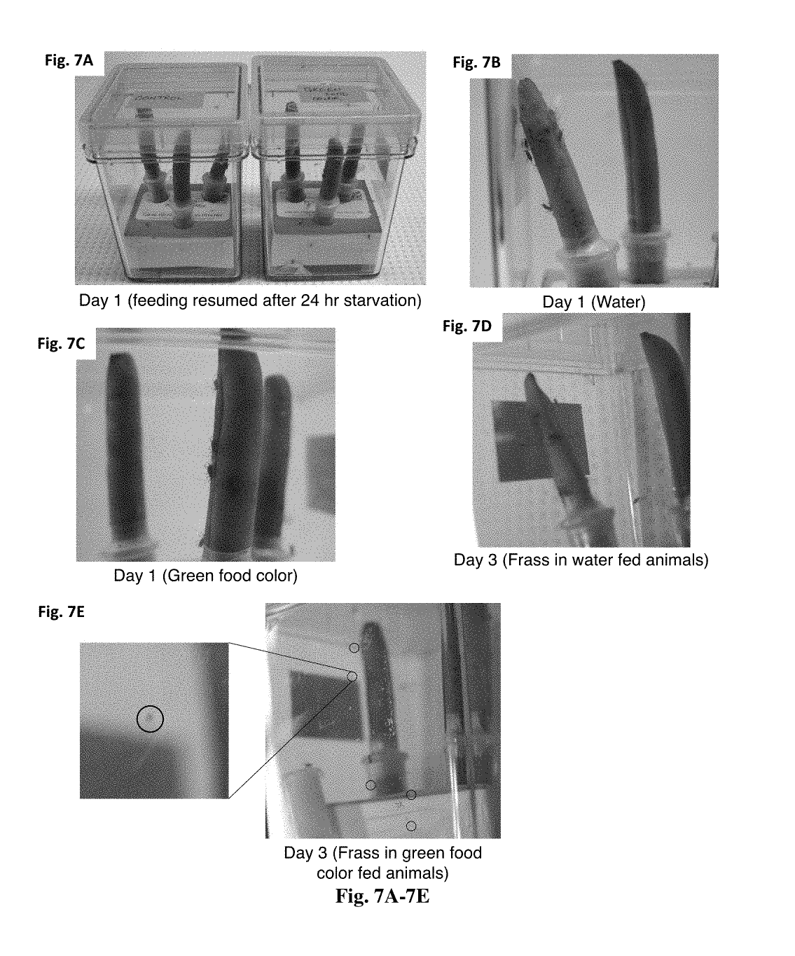

Next we tested the susceptibility to vegetable mediated delivery in other hemipteran insects such as the harlequin bug (M. histrionica) (HB) and pea aphids (A. pisum) both of which are known agricultural insect pests. A similar approach to that shown in FIG. 1 was taken to feed these insects with a solution containing green food color mediated through green beans. Our observations revealed that both these insects were capable of surviving on the green bean diet (FIGS. 6A-H and 7A-E). This was evident from the green frass that is evidence of dietary ingestion from the green beans (FIGS. 6G, 6H, and 7E). These results suggest that our dsRNA-laden vegetable delivery protocol is available for other insects to deliver treatments such as dsRNA for insect biocontrol.

Although these pests are not natural predators for green beans they were observed to feed on them. Though the study was focused on BMSB, delivery methods to other insects such as the HB and pea aphids was successfully tested using green beans as a delivery system. However, not all insects would be expected to feed on green beans, so the applicability of this novel approach using a different plant structure—young collard green leaves—was tested.

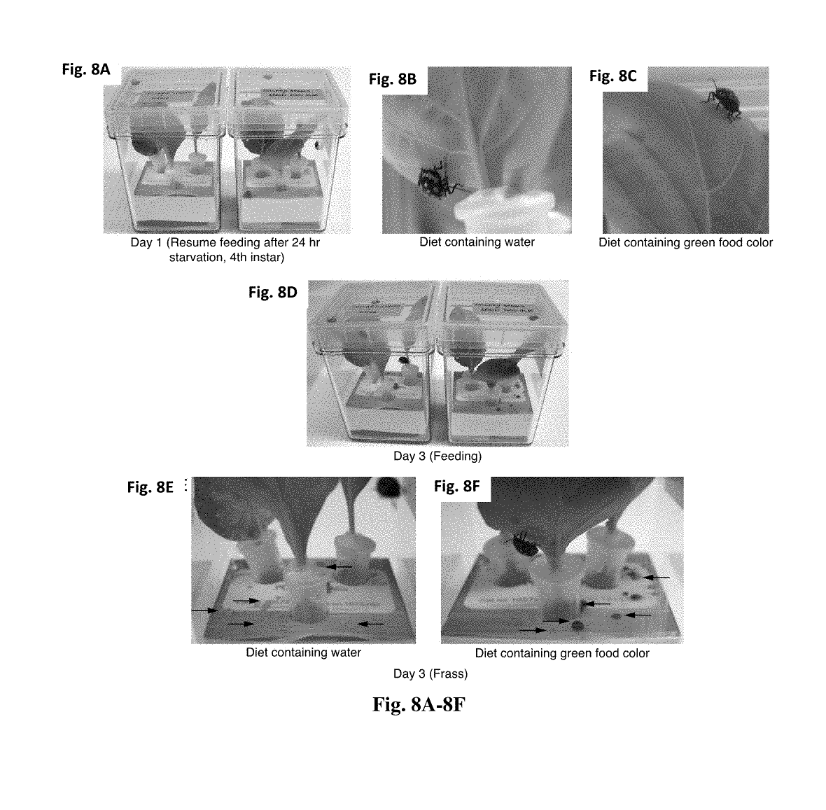

Collard greens (B. oleracea var. viridis) were analyzed as a vegetable vehicle for dsRNA delivery to HB (FIG. 8A) and the leaves were prepared in a similar manner to that described for the green beans used to test that system for BMSB feeding (see above). HBs are a known pest of cole crops such as crucifers or brassicas and infestation causes white stipples due to its piercing/sucking mode of feeding. Efficient uptake of both ddH₂O and a solution of ddH2O with green food color by the HB were apparent from the green colored frass observed on day 3 of the feeding assay (FIGS. 8D, 8E and 8F). These findings show that the approach for targeting sap-feeding pest species can be altered by changing the vegetative structure used to attract the insects.

One of skill in the art will recognize that the disclosed system for dsRNA delivery can be utilized in many other vegetables, fruits, leaves, stems, flowers, and other living plant structures that can uptake and distribute dsRNAs via vascular flow or osmosis. The chosen plant structure can be varied for the type and/or preference of a targeted insect pest to create efficient traps. The ability to create and test the efficacy of such traps is rapid and inexpensive.

While the invention has been described with reference to details of the illustrated embodiments, these details are not intended to limit the scope of the invention as defined in the appended claims. The embodiment of the invention in which exclusive property or privilege is claimed is defined as follows:

What is claimed is:

1. A composition comprising a living plant material and at least one double-strand RNA (dsRNA) not produced by the living plant material, wherein the at least one dsRNA is distributed throughout at least part of the living plant material's vascular tissues and wherein the living plant material does not contain genetic information allowing for the production of the at least one double strand dsRNA.

2. The composition of claim 1, wherein the living plant material is a fruit, vegetable, stem or leaf.

3. The composition of claim 1, wherein the living plant material is a green bean or collard green leaf.

4. The composition of claim 1, wherein the at least one dsRNA is capable of interfering with polypeptide production in at least one insect.

5. The composition of claim 4, wherein the at least one insect is a sap-feeding insect.

6. The composition of claim 5, wherein the sap-feeding insect is a brown marmorated stink bug, a harlequin bug or a pea aphid.

7. The composition of claim 1, wherein the at least one dsRNA comprises two or more distinct dsRNA species.

8. The composition of claim 1, wherein the at least one dsRNA is present at a concentration of 1-2 μg per inch of the living plant material.

9. The composition of claim 1, wherein the at least one dsRNA is introduced to said living plant material by soaking a portion of the living plant material in an aqueous solution comprising the at least one dsRNA.

10. The composition of claim 9, wherein the aqueous solution contains the at least one dsRNA at a concentration of 2-10 μg/ml.

11. A method of inducing RNA interference (RNAi) in an insect, comprising the steps of:
a) providing a living plant material containing at least one double-strand RNA (dsRNA) not produced by the living plant material, wherein the at least one dsRNA is distributed throughout at least part of the living plant material's vascular tissues and wherein the living plant material does not contain genetic information allowing for the production of the at least one dsRNA;
b) allowing the insect to ingest a sufficient amount of the at least one dsRNA by feeding on the plant material to interfere with the production of at least one protein targeted by the at least one dsRNA, thereby inducing RNAi in the insect.

12. The method of claim 11, wherein the at least one dsRNA is present at a concentration of 1-2 μg per inch of the living plant material.

13. The method of claim 11, wherein the living plant material is a fruit, vegetable, stem or leaf.

14. The method of claim 11, wherein the living plant material is a green bean or collard green leaf.

15. The method of claim 11, wherein the induced RNAi is at a level to control the insect.

16. The method of claim 11, wherein the insect is a sap-feeding insect.

17. The method of claim 16, wherein the sap-feeding insect is a brown marmorated stink bug, a harlequin bug or a pea aphid.

18. The method of claim 11, wherein the at least one dsRNA comprises two or more distinct dsRNA species.

19. The method of claim 18, wherein the two or more distinct dsRNA species target protein production in two or more insects.

20. The method of claim 11, wherein the at least one dsRNA is introduced into said living plant material by soaking a portion of the living plant material in an aqueous solution comprising the at least one dsRNA.

21. The method of claim 20, wherein the aqueous solution contains the at least one dsRNA at a concentration of 2-10 μg/ml.

22. The method of claim 20, wherein the living plant material is provided to the insect within six days of introducing the at least one dsRNA into the living plant material.

23. A method of controlling an insect comprising the steps of:
a) providing a living plant material containing at least one double-strand RNA (dsRNA) not produced by the living plant material, wherein the at least one dsRNA is distributed throughout at least part of the living plant material's vascular tissues and wherein the living plant material does not contain genetic information allowing for the production of the at least one dsRNA;
b) allowing the insect to ingest a sufficient amount of the at least one dsRNA, by feeding on the plant material, to interfere with the production of at least one protein targeted by the at least one dsRNA, thereby inducing RNAi in the insect, and;
c) controlling the insect via RNAi.

24. The method of claim 23, wherein the at least one dsRNA is present at a concentration of 1-2 μg per inch of the living plant material.

25. The method of claim 23, wherein the living plant material is a fruit, vegetable, stem or leaf.

26. The method of claim 23, wherein the living plant material is a green bean or collard green leaf.

27. The method of claim 23, wherein the insect is a sap-feeding insect.

28. The method of claim 27, wherein the sap-feeding insect is a brown marmorated stink bug, a harlequin bug or a pea aphid.

29. The method of claim 23, wherein the at least one dsRNA comprises two or more distinct dsRNA species.

30. The method of claim 29, wherein the two or more distinct dsRNA species target protein production in two or more insects.

31. The method of claim 23, wherein the at least one dsRNA is introduced into said living plant material by soaking a portion of the living plant material in an aqueous solution comprising the at least one dsRNA.

32. The method of claim 31, wherein the aqueous solution contains the at least one dsRNA at a concentration of 2-10 μg/ml.

33. The method of claim 31, wherein the living plant material is provided to the insect within six days of introducing the at least one dsRNA into the living plant material.
